(19) United States
(12) Patent Application Publication
(10) Pub. No.: US 2008/0087279 A1
(43) Pub. Date: Apr. 17, 2008

(54) Metered dose inhaler

(76) Inventors: Catharine Laureen Johnson Tieck, Huxley, IA (US); Kenneth William Johnson, Colorado Springs, CO (US)

Correspondence Address: HOLLAND & HART, LLP, P.O BOX 8749, DENVER, CO, 80201, US

(21) Appl. No.: 11/546,224

(22) Filed: Oct. 11, 2006

(51) Int. Cl. A61M 11/00; A61M 15/00

(52) U.S. Cl. 128/200.23; 128/203.15

(57) ABSTRACT

There is disclosed a metered dose inhaler for administering medication. In an embodiment, the metered dose inhaler includes a housing, a medication canister, a mouth-piece, an extension forming a chamber within the housing, and a one-way valve. In one embodiment, the metered dose inhaler includes a flange disposed on the mouth-piece. In another embodiment, the metered dose inhaler includes a peripheral edge configured to reduce spray away from the mouth-piece. In an embodiment, the metered dose inhaler includes a telescoping articulation between first and second ends of the housing. In another embodiment, the metered dose inhaler includes a telescoping extension. Other embodiments are also disclosed.

### BACKGROUND

Referring to FIGS. 13A, 13B and 14, there are shown various illustrations of the human anatomy. In each of these illustrations, lungs 1300 are shown. In FIG. 13A, lungs 1300 are shown together with other parts of the respiratory system, including nasal cavity 1302, oral cavity 1304, trachea 1308, and bronchus 1310. In FIG. 13B, there is shown a muscle strand 1312 adjacent to terminal bronchiole 1314. Also shown is alveolus 1316 and alveolar capillaries 1318. FIG. 15 illustrates lungs 1300 with bronchioles 1314 and alveoli 1316.

Any medication that deposits in bronchioles 1314 has a lock/key effect and releases smooth muscles 1312 that are constricting the airways. The only way the medication can be delivered to the desired airways is to have a slow deep breath. This allows the medication to deposit on the airways as it passes through them. The medication as it is dispensed varies in micron sizes from 0.5 microns to 25 microns. This could be considered equivalent to boulders and peas. The larger sized particles, in microns and in weight, will fall quickly and smaller ones may miss bronchioles 1314 and go to distal parts of the lungs 1300, i.e., alveoli 1316, where there is minimal therapeutic affect.

One typical problem with delivery of medication is the mouth. This is a highly vascular area and any medication landing in that area will be absorbed into the blood. This causes the majority of the undesired side effects, including cardiac issues and tremors. Because the flow is fast coming out of the typical metered dose inhaler, it is often difficult for a flow challenged person, such as an asthmatic, to match the flow rate, and often more than 50% of the medication deposits in the mouth and throat.

Coordination of inhalation and actuation of the metered dose inhaler is often a serious problem. People often get partial doses and waste many doses due to poor coordination and technique. A typical metered dose inhaler needs to be usable for pediatric and geriatric age groups. With strength and agility as a factor, partial doses and ineffective doses are frequent problems.

Currently, the U.S. Environmental Protection Agency (EPA) allocates a set amount CFCs for use with metered dose inhalers due to the affect on the ozone layer. By 2008, all metered dose inhalers (MDIs) must be free of CFCs. Some MDIs have made the change to the CFC-free delivery systems.

It is estimated that there are more than 300 million asthma-related sufferers according to the World Health Organization. The most common medicine used for asthma is a beta-2 agonist, commonly known as albuterol, delivered through a metered dose inhaler. It is considered a “rescue” drug because it works within 5 minutes.

According to the EPA and the CDC, more than 60 million albuterol inhalers were sold in the United States by the Apr. 21, 2006 meeting. In this meeting, the EPA and pharmaceutical companies representing the metered dose inhaler producers stated that nationally we are facing an imminent crisis in a shortage of “rescue” metered dose inhalers.

One of the biggest problems with the current metered dose inhaler now using a CFC is a build-up of the drug at the spray opening. This tends to clog the hole and give inconsistent and ineffective doses to the user. Often the user will assume the metered dose inhaler is empty and replace it. Additionally, the environment has a big factor. Moisture, humidity, and dirt debris can get in the container and have the same affect.

Another problem is that CFCs that were intended to be used for the production of metered dose inhalers were allocated to a non-producing company. This in turn has placed a huge restriction on production capability of metered dose inhalers.

Asthmatics may be treated with “rescue” medicines and maintenance medications. The current metered dose inhaler, albuterol is used as a “rescue” medication, but has been used as both a “rescue” medicine and a maintenance medicine for years.

There are several problems with delivery and deposition of the current metered dose inhaler medications. The average person with good technique gets about half the “intended dose”. With proper technique and use of a spacer or a chamber, studies show 10-14% delivery per actuation. The American Lung Association, Centers for Disease Control (CDC), the Food and Drug Administration (FDA) recommends a spacer as the best way to get a good delivery of the medication. However, most asthmatic and chronic obstructive pulmonary disease (COPD) patients are non-compliant with the use of spacers. This may be in part due to the lack of education and resources. However, often, it is simply a result of inconvenience.

Studies have shown that the average asthmatic uses their MDI as a “rescue” drug as often as three times per week. Many asthmatics are active, athletic, and have very busy lifestyles. They may have been educated to use their maintenance dose prescription one to two times per day so as to have a longer duration in their system. However, these are not a fast-acting drugs and may take a long time for the full affect, and may not replace, or be used as, a “rescue” medication. People often use their “rescue” MDI before exercise or before possible exposure to “triggers.” That can induce an attachment and increase the need for “rescue.”

The downside is that these people are “flow” challenged. They cannot typically take a quick-deep breath to match the delivery of the inhaler because the majority of the medication deposits in the mouth and throat as a result. This is what a spacer is designed to prevent. A chamber, or a two finger distance, between the end of the metered dose inhaler and the mouth of the user, drastically improves delivery of the medication and the effort needed by the patient.

Expected changes for the new CFC-free metered dose inhaler require modifications in the design of the aerosol delivery. Although this solves the problem for delivery of the medication without the use of a CFC, it does not change the difficulty for the asthmatic to get a full dose especially in a “rescue” situation. This is because the new design of the CFC-free metered dose inhaler causes a “push” at a higher rate, which in turn means a faster push for the user to match. As such, the user has an increased need for a faster and deeper breath.

One remedy is the use of a spacer. However, a spacer added to an MDI changes its ability to be small, portable, compact, discrete, and convenient. An executive is not generally willing to keep a spacer in his or her suit. A runner will not typically have a pocket or pouch large enough to carry a spacer. Furthermore, patients often opt not to use a spacer because it draws additional attention due to its size. Another problem is access. The spacer must be stored in a place where it is accessible enough in a “rescue” situation. Often, it is kept in an inconvenient location.

Another alternative is a breath demand valve. One problem with the typical alternative breath demand valve is a greater chance of contamination occurring in more than two places within the delivery valve system. Another problem is that flow-challenged asthmatic users in a rescue situation may not be able to maintain a slow deep breath long enough to receive the medicine. This is a typical push versus. pull scenario. Still another problem is that the demand for flow has to be easy and universal for all users of all age groups. One other problem is the demand valve be subject to the previous environmental factors such as moisture, humidity, and prescription build-up that may render the valve in a fixed position. Also, contamination may slow or delay the valve which may lead to a varying dose effect.

In addition, MDIs are classified by the FDA as drugs, and are therefore dispensed by pharmacies. Spacers are unfortunately classified as durable medical equipment and are dispensed by durable medical equipment suppliers. Often these suppliers are located a different location than a patients pharmacy. Also, as durable medical equipment, the spacers are sometimes not covered by insurance plans. In other instances, doctors forget to write a separate prescription for the durable medical equipment vendor.

### SUMMARY OF THE INVENTION

In an embodiment, there is provided a metered dose inhaler for administering medication, the metered dose inhaler comprising a housing having a first end and a second end, the first end being configured to hold a medication canister that is actuated to selectively administer medication through the housing and out of the second end; a mouth-piece disposed adjacent the second end of the housing; an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and a one-way valve disposed adjacent the second end of the housing.

In another embodiment, there is provided a metered dose inhaler for administering medication, the metered dose inhaler comprising a housing having a first end and a second end, the first end being configured to hold a medication canister that is actuated to selectively administer medication through the housing and out of the second end; a mouth-piece disposed adjacent the second end of the housing; an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and a flange disposed on the mouth-piece, between the chamber and the second end of the housing, and the flange configured a distance away from the second end of the housing to encourage the user to put teeth and lips around the mouth-piece, and to prevent occlusion of the mouth-piece with the teeth and lips.

In yet another embodiment, there is provided a metered dose inhaler for administering medication, the metered dose inhaler comprising a housing having a first end and a second end, the first end being configured to hold a medication canister that is actuated to selectively administer medication through the housing and out of the second end; a medication canister disposed at the first end of the housing, the medication selectively administered through the housing and out of the second end; a mouth-piece disposed adjacent the second end of the housing; an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and a peripheral edge extending inwardly from the housing adjacent to the medication canister, the peripheral edge configured to reduce a spray of the medication through the second end of the housing.

In still another embodiment, there is provided a metered dose inhaler for administering medication, the metered dose inhaler comprising a housing having a first end and a second end, the first end being configured to hold a medication canister that is actuated to selectively administer medication through the housing and out of the second end; a mouth-piece disposed adjacent the second end of the housing; an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and a telescoping articulation between the first end and the second end of the housing, a first portion of the housing, between the articulation and the first end, having the medication canister therein, a second portion of the housing, between the articulation and the second end, having the mouth-piece and the extension, and the first portion and the second portion configured to articulate with respect to one another to slide together to position the second portion in a surrounding configuration with the first portion for storage, and to slide apart from one another and articulate with respect to one another, to position the mouth-piece of the second portion away from the medication canister within the first portion for use.

In another embodiment, there is provided a metered dose inhaler for administering medication, the metered dose inhaler comprising a housing having a first end and a second end, the first end being configured to hold a medication canister that is actuated to selectively administer medication through the housing and out of the second end; a mouth-piece disposed adjacent the second end of the housing; a telescoping extension forming a chamber within the housing, between i) where a nozzle of the medication canister is positioned when the medication canister is held by the first end of the housing, and ii) the mouth-piece, wherein the telescoping extension has a variable length from the medication canister to the mouth-piece, a maximum length of the telescoping extension configured to optimize delivery of the medication from the medication canister to lungs of a user, and a minimum length of the telescoping extension configured to optimize portability of the metered dose inhaler; and a one-way valve disposed adjacent the second end of the housing, between the chamber and the mouth-piece.

In yet another embodiment, there is provided a metered dose inhaler for administering medication, the metered dose inhaler comprising a housing having a first end and a second end, the first end being configured to hold a medication canister that is actuated to selectively administer medication through the housing and out of the second end; a mouth-piece disposed adjacent the second end of the housing; a telescoping extension forming a chamber within the housing, between i) where a nozzle of the medication canister is positioned when the medication canister is held by the first end of the housing, and ii) the mouth-piece, wherein the telescoping extension has a variable length from the medication canister to the mouth-piece, a maximum length of the telescoping extension configured to optimize delivery of the medication from the medication canister to lungs of a user, and a minimum length of the telescoping extension configured to optimize portability of the metered dose inhaler; and a flange disposed on the mouth-piece, between the chamber and the second end of the housing, and the flange configured a distance away from the second end of the housing to encourage the user to put teeth and lips around the mouth-piece, and to prevent occlusion of the mouth-piece with the teeth and lips.

In still another embodiment, there is provided a metered dose inhaler for administering medication, the metered dose inhaler comprising: a housing having a first end and a second end, the first end being configured to hold a medication canister that is actuated to selectively administer medication through the housing and out of the second end; a mouth-piece disposed adjacent the second end of the housing; a telescoping extension forming a chamber within the housing, between i) where a nozzle of the medication canister is positioned when the medication canister is held by the first end of the housing, and ii) the mouth-piece, wherein the telescoping extension has a variable length from the medication canister to the mouth-piece, a maximum length of the telescoping extension configured to optimize delivery of the medication from the medication canister to lungs of a user, and a minimum length of the telescoping extension configured to optimize portability of the metered dose inhaler; and a peripheral edge extending inwardly from the housing adjacent to the medication canister, the peripheral edge configured to reduce a spray of the medication through the second end of the housing.

In an embodiment, there is disclosed a metered dose inhaler for administering medication, the metered dose inhaler comprising a housing having a first end and a second end, the first end being configured to hold a medication canister that is actuated to selectively administer medication through the housing and out of the second end, and the first end having a v-shaped groove formed therein; a mouth-piece disposed adjacent the second end of the housing; and an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user.

In another embodiment, there is disclosed a metered dose inhaler for administering medication, the metered dose inhaler comprising a housing having a first end and a second end, the first end being configured to hold a medication canister that is actuated to selectively administer medication through the housing and out of the second end; a lip extending outwardly from at least a portion of the housing adjacent the first end thereof, and adjacent an outwardly extending portion of the canister when positioned in the first end of the housing; a mouth-piece disposed adjacent the second end of the housing; and an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user.

Other embodiments are also disclosed.

### BRIEF DESCRIPTION OF THE DRAWINGS

Illustrative embodiments of the invention are illustrated in the drawings, in which:

FIG. 1 illustrates a novel metered dose inhaler having an extension forming a chamber for optimizing the administration of medication, a one-way valve at the end of the chamber, near the mouth-piece, and a flange for placement of teeth and lips on the mouth-piece;

FIGS. 2A-2E illustrate one embodiment of a novel metered dose inhaler having a canister holding portion and an extension portion configured to articulate and telescope with respect to one another;

FIGS. 3A and 3B illustrates one exemplary embodiment of a novel metered dose inhaler having a telescoping extension, a one-way valve at the end of the chamber, near the mouth-piece, and a flange for placement of teeth a lips on the mouth-piece;

FIG. 4 illustrates a novel metered dose inhaler having a canister configured to spray in a direction away from the mouth-piece, a rear wall extending an additional distance away from the mouth-piece, and a ring within the chamber to moderate the flow rate of mediation being administered;

FIG. 5 illustrates a novel metered dose inhaler with a corkscrew passageway configured through the chamber to moderate the flow rate of medication being administered;

FIG. 6 illustrates a novel metered dose inhaler with a set of diverters within the chamber to moderate the flow rate of medication being administered;

FIG. 7 illustrates another exemplary embodiment of one embodiment of a novel metered dose inhaler having a canister holding portion and an extension portion configured to articulate and telescope with respect to one another; which is shown in the use configuration;

FIGS. 7A and 7B illustrate cross-sectional views of two embodiments of the metered dose inhaler shown in FIG. 7;

FIG. 8A illustrates the extension portion, which is shown in FIG. 7, disconnected from the canister holding portion;

FIG. 8B illustrates a cross-sectional view of the extension portion, which is shown in FIG. 8;

FIG. 9 illustrates the canister holder portion, which is shown in FIG. 7, disconnected from the extension portion;

FIG. 10 illustrates the metered dose inhaler shown in FIG. 7, in an intermediate position between a configuration for use and a configuration for storage;

FIG. 10A illustrates a cross-section view of the metered dose inhaler as shown in FIG. 10;

FIG. 11 illustrates the metered dose inhaler shown in FIG. 7, in positioned in a configuration for storage;

FIG. 11A illustrates a cross-sectional view of the metered dose inhaler as shown in FIG. 11;

FIG. 12 illustrates another exemplary embodiment of one embodiment of a novel metered dose inhaler having a canister holding portion and an extension portion configured to articulate and telescope with respect to one another; which is shown in the use configuration;

FIG. 12A illustrates a cross-sectional view of the metered dose inhaler as shown in FIG. 12; and

FIGS. 13A, 13B and 14 illustrate anatomy of the lungs.

### DETAILED DESCRIPTION

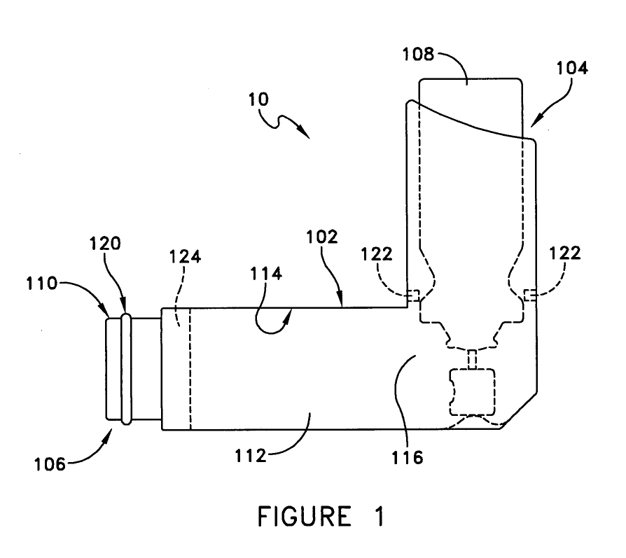

Referring to FIGS. 1, 2A, 2B, 2C, 3A, 3B, 4-7, 10, 11 and 12, there is illustrated several embodiments of novel metered dose inhalers 100 for administering medication. In an embodiment, the medication may include a bronchodialator, which may be albuterol. In another embodiment, the medication may include one or more maintenance drugs, which may include a steroidal drug such as flovent. In other embodiments, the medication may include other inhalable drugs now known or discovered in the future.

In various embodiments, novel metered dose inhaler 100 may include a housing 102 having a first end 104 and a second end 106. A medication canister 108 may be disposed at first end 104 of housing 102. Medication may be selectively administered through housing 102 and out of second end 106.

A mouth-piece 110 may be disposed at second end 106 of housing 102. An extension 112 may form a chamber 114 within housing 102, between medication canister 108 and mouth-piece 110. Extension 112 may have a length from medication canister 108 to mouth-piece 110 configured to optimize delivery of the medication from medication canister 108 to lungs of a user.

Generally, a long extension slows delivery of medication to allow better breath coordination and delivery. Extension 112 may also help to keep the interior portions clean and preserves working parts. This may minimize clogging and obstruction at or near medication canister 108. Extension 112 also allows the larger particles, which are referred to as “boulders” above, to drop out of the airflow prior to delivery of the medication into the user's mouth.

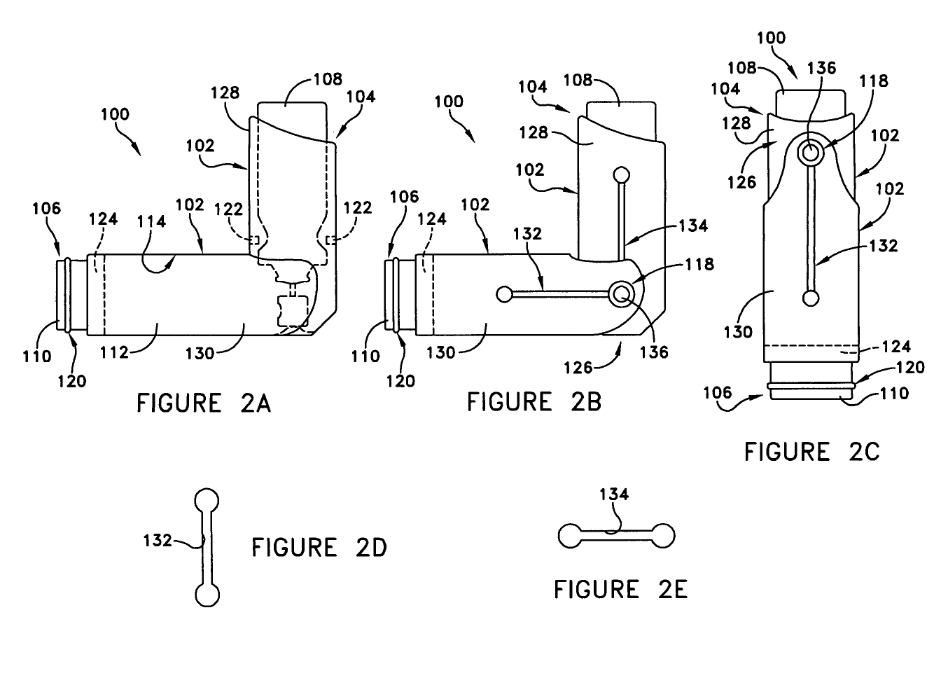

Referring to FIGS. 1 and 4-6, and in one embodiment, housing 102 is a single, integral body 116 from first end 104 to the second end 106 of metered dose inhaler 100. The integral body 116 may be formed, for example, by an injection molding process using plastic. Referring now to FIGS. 2A-2C, 7, 7A, 7B, 10, 10A, 11, 11A, 12 and 12A, and in another embodiment, housing 102 may include an articulation 118 between first end 104 and second end 106.

In an embodiment, a flange 120 may be disposed on mouth-piece 110. Generally, flange 120 is located between chamber 114 and second end 106 of housing 102. Flange 120 may be configured a distance away from second end 106 of housing 102 in order to encourage the user to put his or her teeth and lips around mouth-piece 110, which in turn prevents occlusion of mouth-piece 110 with the user's teeth and lips. For example, flange 120 may include, but is not limited to a protruding rim, edge, rib, or collar extending from mouth-piece 110. Generally, the slope of mouth-piece 110 is interrupted by flange 120 but is relatively constant on either side of flange 120.

Referring to FIGS. 1, 2A, 3A, 3B, 4, 5, 6, 7A, 7B, 10A, 11A and 12A, there is shown a peripheral edge 122 extending inwardly from housing 102 adjacent to medication canister 108. Peripheral edge 122 may be configured to reduce a spray of the medication around medication canister 108, through second end 106 of housing 102.

Looking at FIGS. 1, 2A-2C, 3A, 3B, 4, 5, 6, 7A, 7B, 8B, 10A and 11, and in an embodiment, a one-way valve 124 may be disposed adjacent second end 106 of housing 102, between chamber 114 and mouth-piece 110. One-way valve 124 acts as a one-way door to only expose the inside of the container during inspiration. Because negative flow opens one-way valve 124, there is only minimal, if any, effect on chamber 114 and working parts. Additionally, one-way valve 124 allows the medication to sit in a waiting pattern, and this allows the user to get a full dose with a slow or compromised breath.

Referring to FIGS. 2A-2C, 7, 7A, 7B, 10, 10A, 12 and 12A, a metered dose inhaler 100 may include a telescoping articulation 126 between first end 104 and second end 106 of housing 102. A first portion 128 of housing 102, between articulation 118 and first end 104, may contain medication canister 108. Herein below, first portion 128 may also be referred to as a canister holding portion 128. A second portion 130 of housing 102, between articulation 118 and second end 106, may have mouth-piece 110 and extension 112.

FIG. 1 illustrates a novel metered dose inhaler 100 which may have extension 112 forming chamber 114 for optimizing the administration of medication. A one-way valve 124 may provided at second end 106 of chamber 114, near the mouth-piece 110. An optional flange 120 may be provided for placement of teeth and lips on the mouth-piece 110.

Looking now at FIGS. 2A-2E, there is shown one embodiment of a novel metered dose inhaler 100 having a canister holding portion 128 and an extension portion 112 configured to articulate and telescope with respect to one another. Telescoping articulation 126 may include at least one slot 132, 134 through one or both of first portion 128 and second portion 130, respectively. A pivot 136 may be disposed within one of slots 132, 134 or both of slots 132, 134.

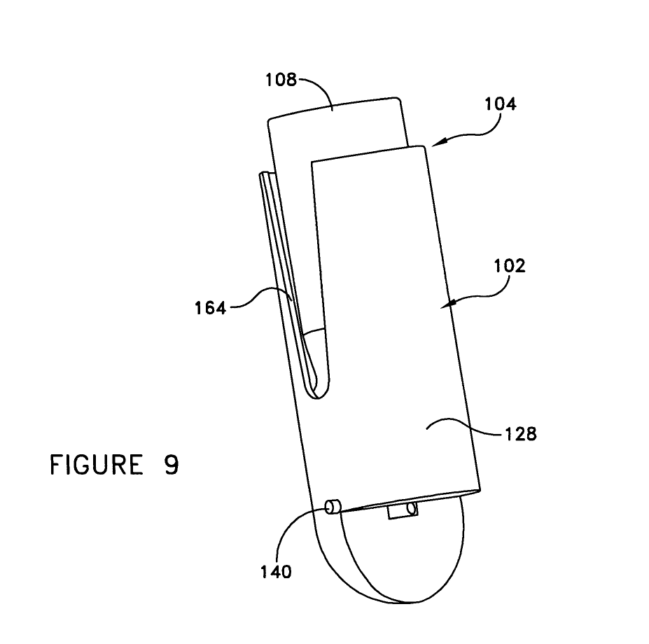

One of first portion 128 and second portion 130 may include a track 138. Referring to FIG. 9, a pivot 140 may extend from one of first portion 128 and second portion 130. Pivot 140 and track 138 may correspond to one another. As shown in FIG. 9, pivot 140 may extend from first portion 128. As shown in FIGS. 7A, 7B, 8A, 1A, 11A and 12A, track 138 is formed within second portion 130. Alternatively, track 138 could be placed on first portion 128 and pivot 140 could be placed on second portion 130.

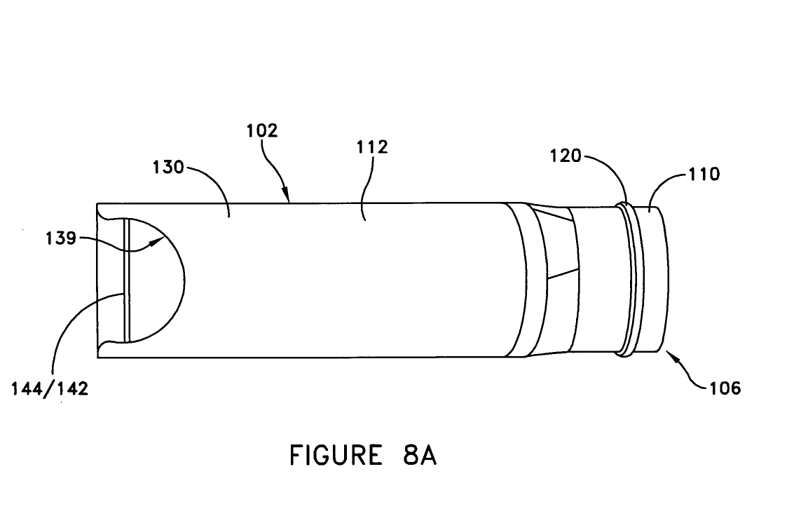

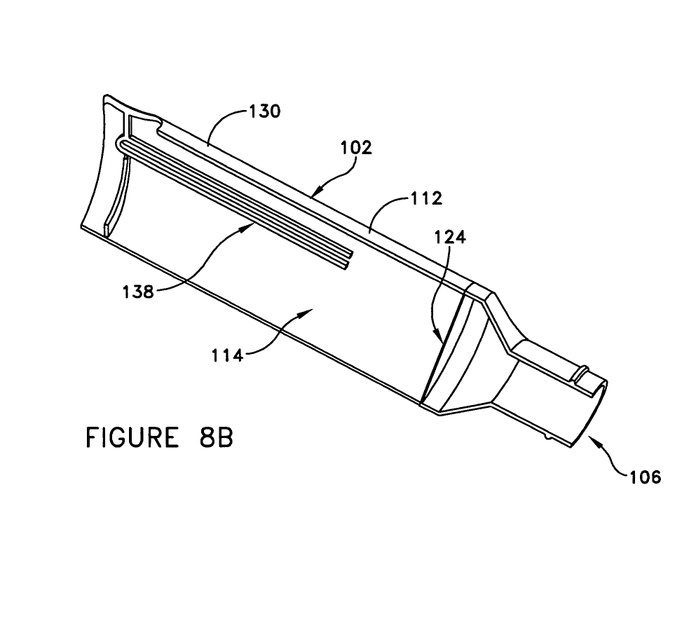

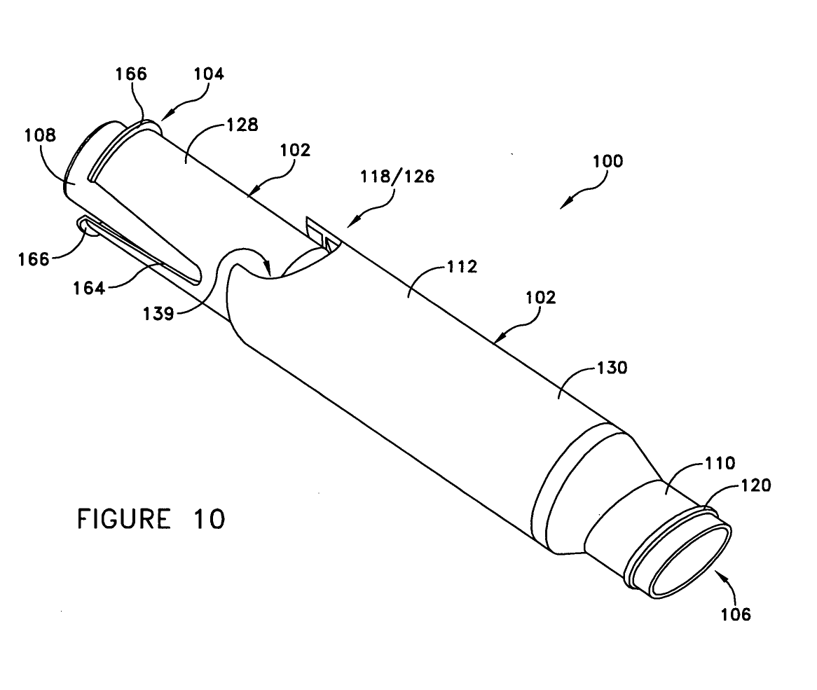

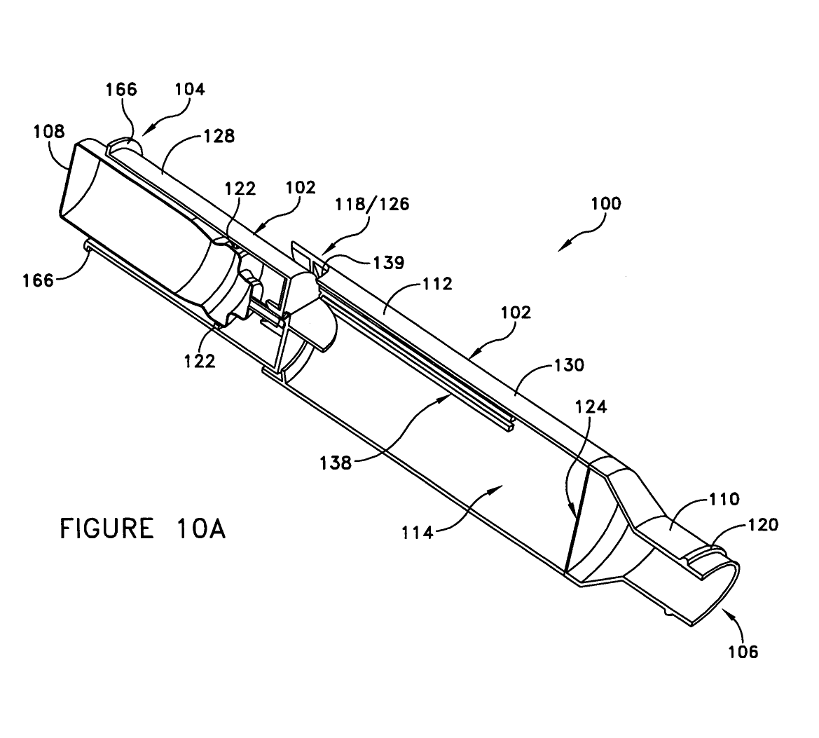

First portion 128 and second portion 130 may be configured to lock with one another to position mouth-piece 110 of second portion 130 away from the nozzle of canister 108 for use. As best illustrated in FIGS. 8A and 10, second portion 130 may include an arcuate opening 139 sized to mate with housing 102 of first portion 128. Furthermore, for example, locking may be accomplished with an attachment device such as projections formed by the shape of opening 139. These projections may be narrower than the diameter of canister holder 128. As such arcuate opening 139 is slightly greater than a semi-circle of 180 degrees so as to create opening 139 as a cord of an imaginary, continuous circle. The length of the cord is less than the diameter of such an imaginary, continuous circle extending through arcuate opening 139. However, it should be appreciated that opening 139 may be other shapes, such as an ellipse. When canister holder 128 is rotated into the upright and locked position, passing through this narrow section, the chamber body 130 flexes slightly and then returns to its shape to hold canister holder 128 in place. When storing holder 128, the reverse is generally true, a small force is required to move holder 128 past the narrow opening 139.

In an embodiment, first portion 128 and second portion 130 may be configured to lock with one another to position second portion 130 in a surrounding configuration with first portion 128 for storage. In one embodiment, first portion 128 and second portion 130 may be sized for frictional engagement with one another to lock with one another.

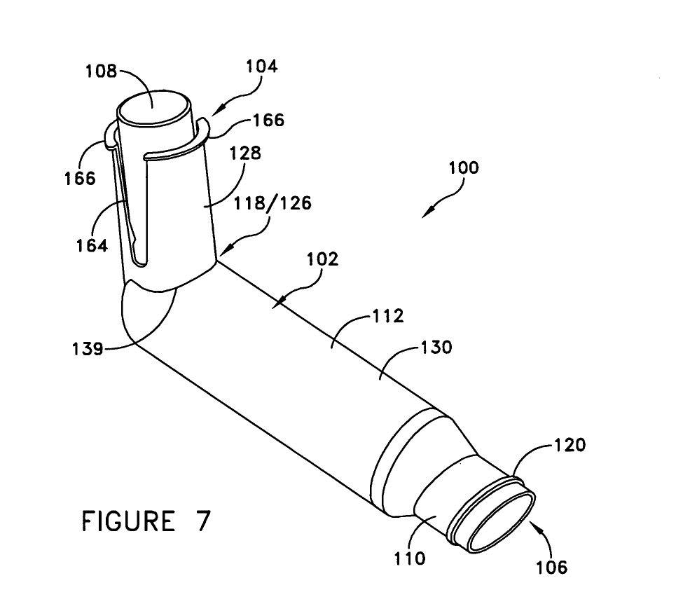

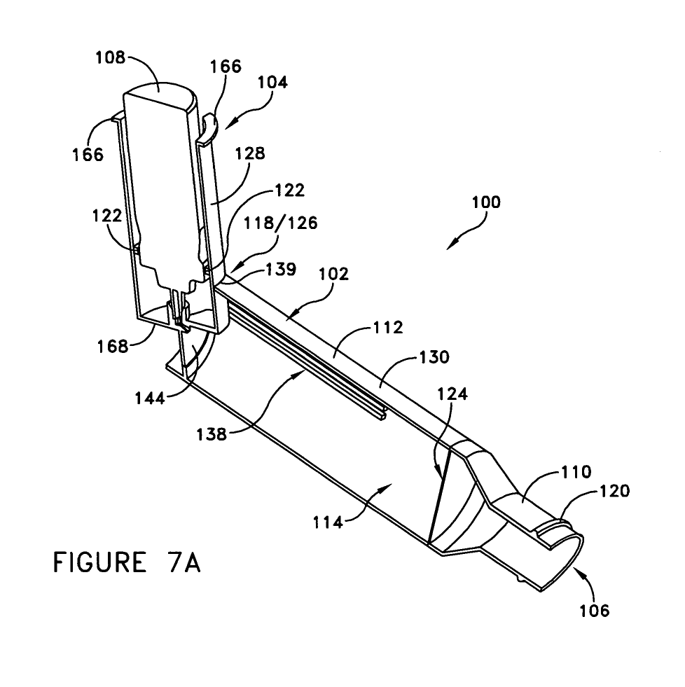

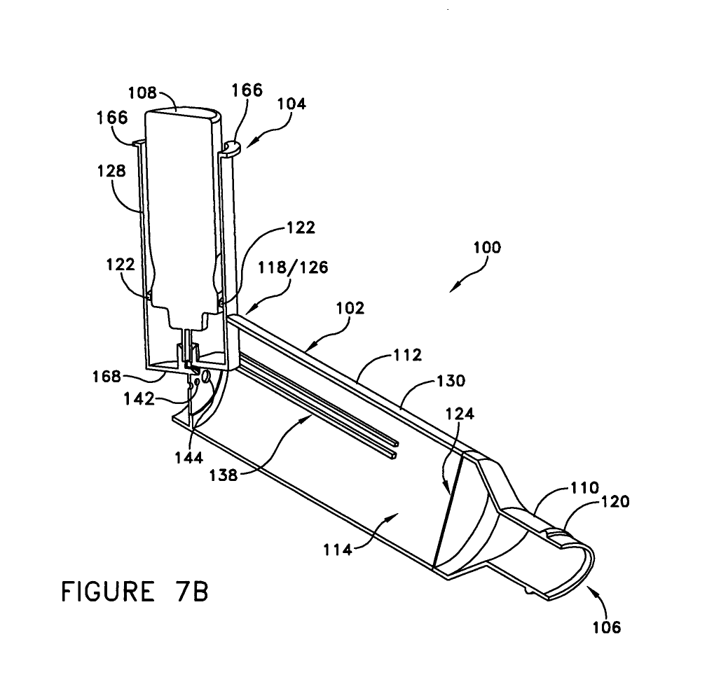

As best illustrated in FIG. 7B, first portion 128 may contain openings 142 adjacent to pivot 140. Such openings 142 may be configured to supply the intake air for when the user inhales buildup into chamber 114 when medication is dispensed from medication canister 108. The size of holes 142 is such that enough area is supplied to make inhalation relatively easy. Without holes 142, it may be similar to trying to suck the air out of a pop bottle. Additionally, in an embodiment, holes 142 may be placed close to the nozzle of medication canister 108 such that the exit velocity of the medication from the nozzle carries the medication away from holes 142. For example, the medicine is not allowed to make a sharp U-turn and go out through the intake holes. In another embodiment, as best illustrated in FIG. 7A, a solid wall 144 may be provided adjacent to pivot 140.

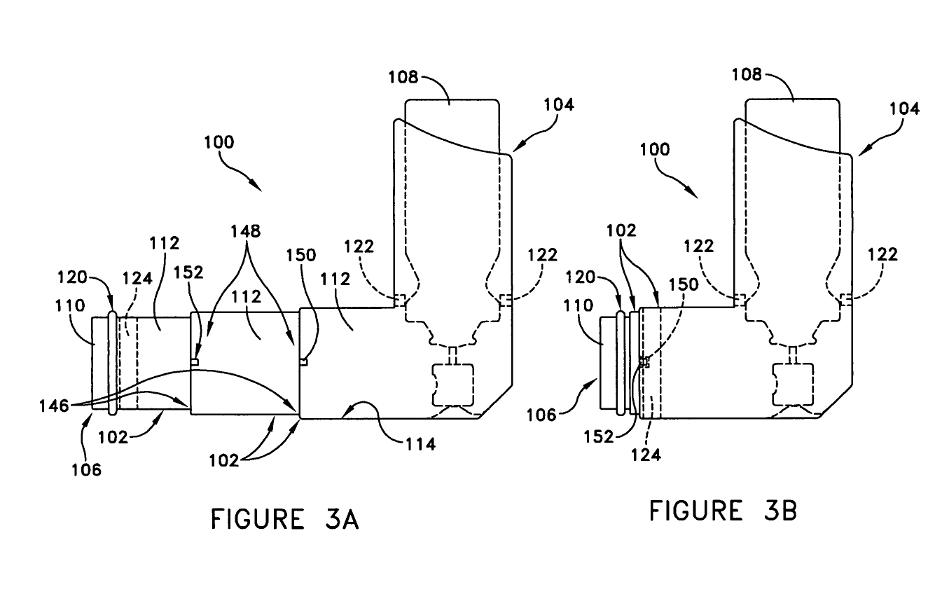

Referring to FIGS. 3A and 3B, and in an embodiment, metered dose inhaler 100 may include a telescoping extension 145 forming a chamber 114 within housing 102, between medication canister 108 and mouth-piece 110. Telescoping extension 145 may have a variable length from medication canister 108 to mouth-piece 110. Telescoping extension 145 may have a maximum length of configured to optimize delivery of medication from medication canister 108 to the lungs of a user. Telescoping extension 145 may have a minimum length configured to optimize portability of metered dose inhaler 100.

Novel metered dose inhaler 100 may include, in addition to telescoping extension 145, one-way valve 124 at second end 106 of the chamber 114, near the mouth-piece 110. Flange 120 may also be included for placement of teeth a lips on the mouth-piece 110.

Telescoping extension 145 may have selectively engaging portions 146 for locking variable length of telescoping extension 112 at maximum length. Telescoping extension 145 may have engaging portions 148 for locking variable length of telescoping extension 112 at minimum length. One of selectively engaging portions 148 may have a recess 150. Another one of selectively engaging portions 148 may have a protrusion 152. Recess 150 and protrusion 152 may be configurable to selectively lock together with one another.

In an embodiment, the configuration of extension 112 with telescoping articulation 126 protects inner components of the novel metered dose inhaler 100 from environmental dirt and debris. In other words, second portion 130 may be configured to help keep dirt and debris out of first portion 128. Extension 112 also serves as a reservoir to hold medication until the user is ready and capable of a slow deep breath. Extension 112 also generally allows improved user and device coordination, which may provide the delivery of the medication as intended by physicians and manufacturers.

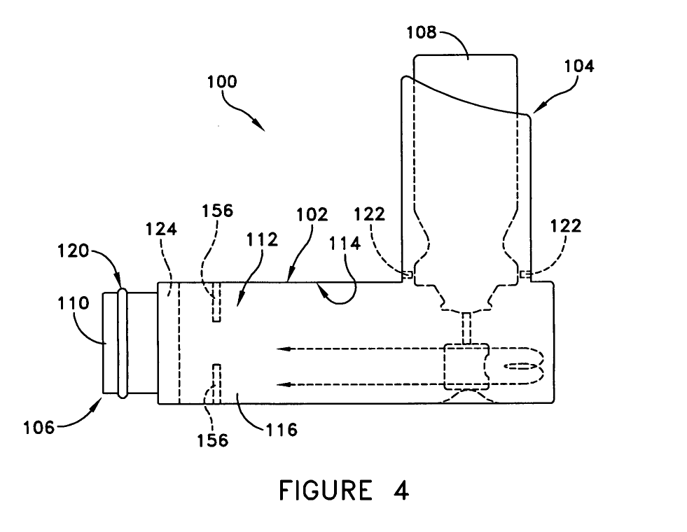

In another embodiment, and referring now to FIG. 4, there is shown a novel metered dose inhaler 100 having medication canister 108 configured to spray in a direction away from the mouth-piece 110. A rear wall 154 may extend an additional distance away from the mouth-piece 110 so as to provide additional room for spray from medication canister 108. A ring 156 may be disposed within the chamber 114. Ring 156 may moderate the flow rate of medication being administered. Ring 156 allows the use of a shortened extension 112.

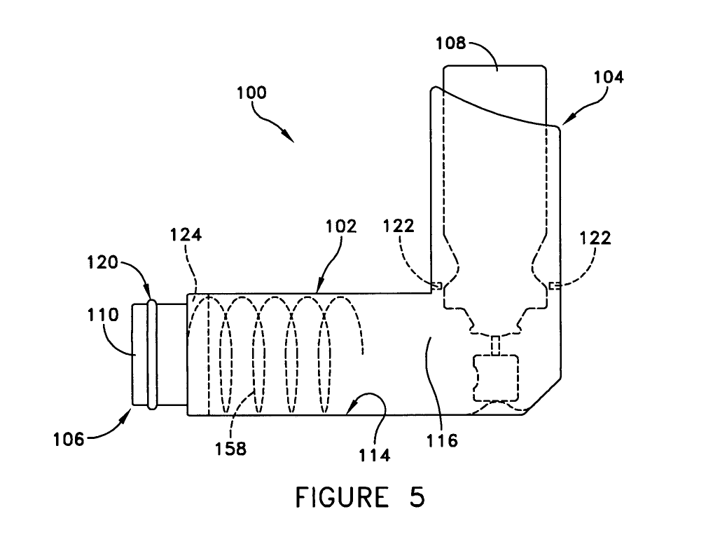

Looking at FIG. 5, there is shown a novel metered dose inhaler 100 with a corkscrew passageway 158 configured through the chamber 114. Corkscrew passageway 158 may moderate the flow rate of medication being administered. Corkscrew passageway 158 allows the use of a shortened extension 112.

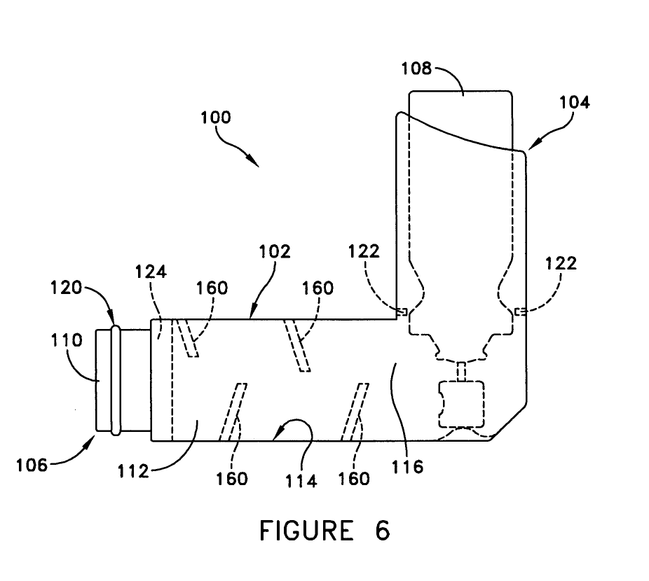

Referring to FIG. 6, and in an embodiment, novel metered dose inhaler 100 may include a set of diverters 160 within the chamber 114. Diverters 160 may be configured to moderate the flow rate of medication being administered. Diverters 160 allow the use of a shortened extension 112.

FIG. 7 illustrates another exemplary embodiment of novel metered dose inhaler 100 having a canister holding portion 128 and an extension portion 112 configured to articulate and telescope with respect to one another. In FIG. 7, metered dose inhaler 100 is shown in the use configuration.

As shown in FIG. 8, extension portion 112 is disconnected from the canister holding portion 128. A cross-sectional view of the extension portion 112 is shown in FIG. 8A. In FIG. 9, there is shown a view of canister holder portion 128 disconnected from the extension portion 112.

FIG. 10 illustrates metered dose inhaler 100, as shown in FIG. 7, in an intermediate position, between an open configuration for use and a closed configuration for storage. FIG. 10A illustrates a cross-section view of the metered dose inhaler 100 as shown in FIG. 10.

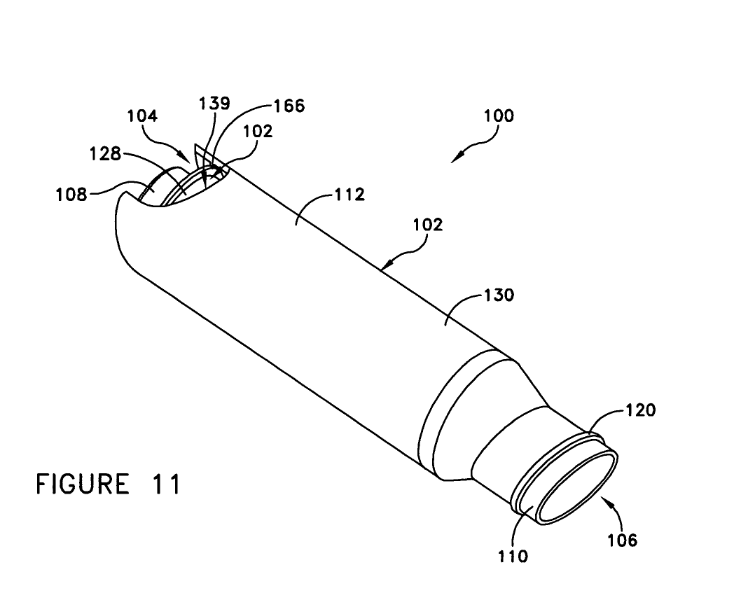

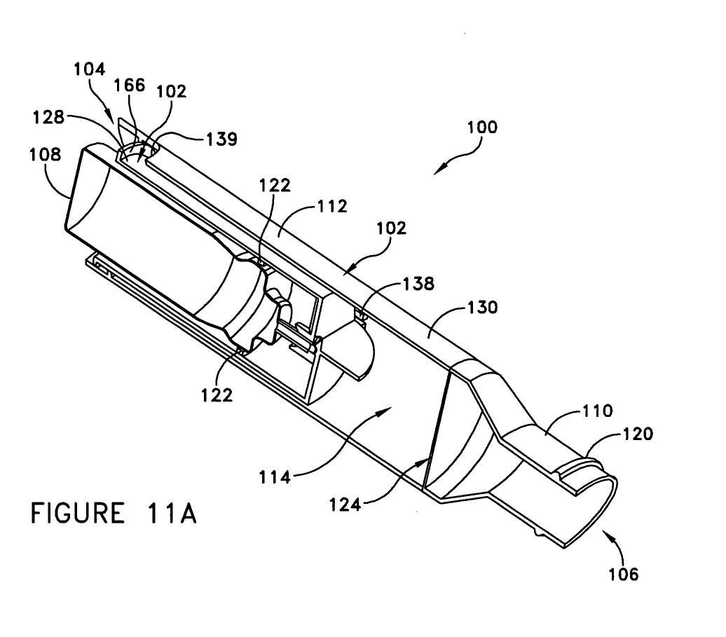

FIG. 11 illustrates metered dose inhaler 100, as shown in FIG. 7, in an closed configuration, positioned for storage. FIG. 11A illustrates a cross-sectional view of the metered dose inhaler 100 as shown in FIG. 11.

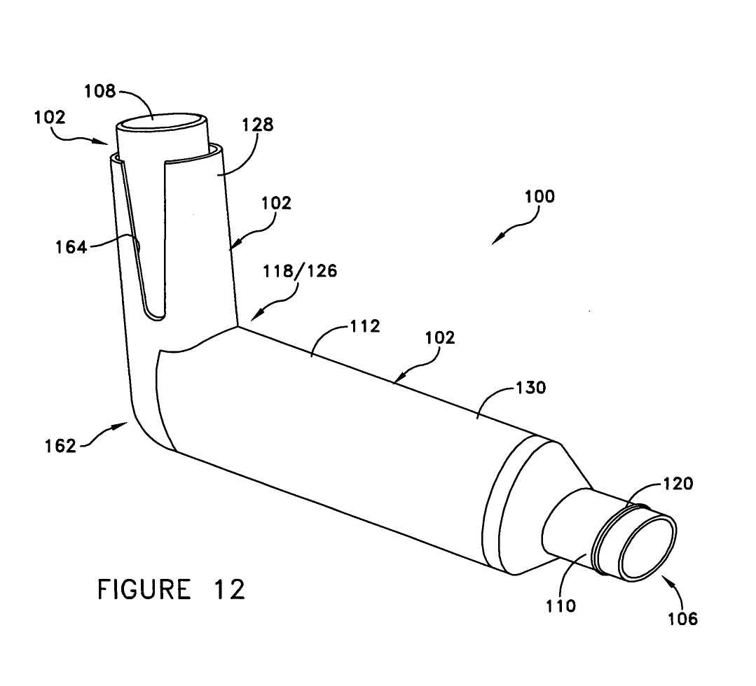

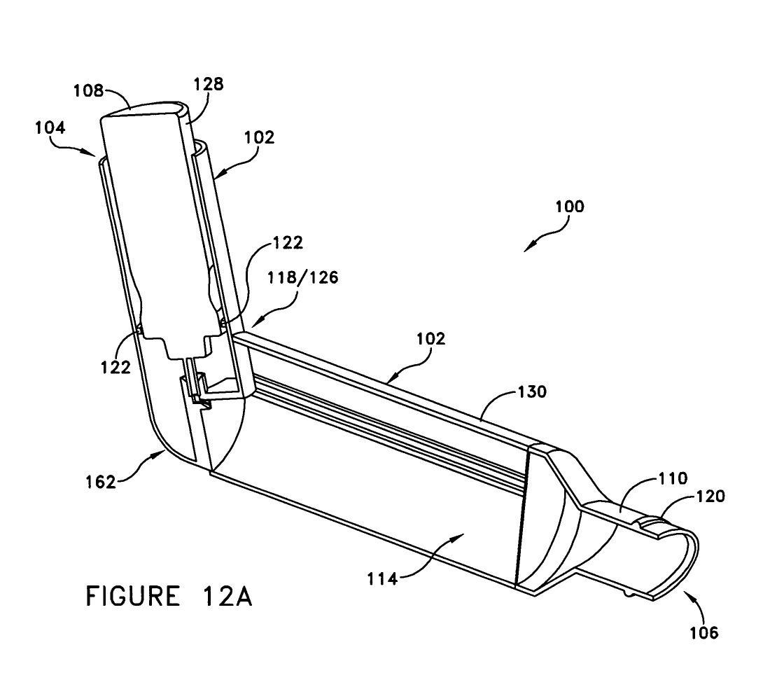

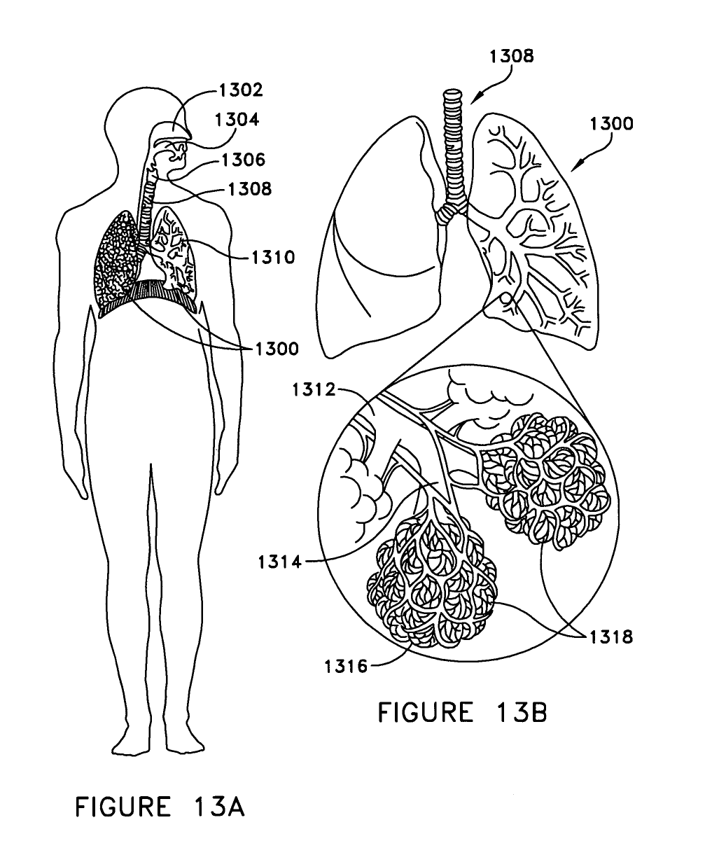

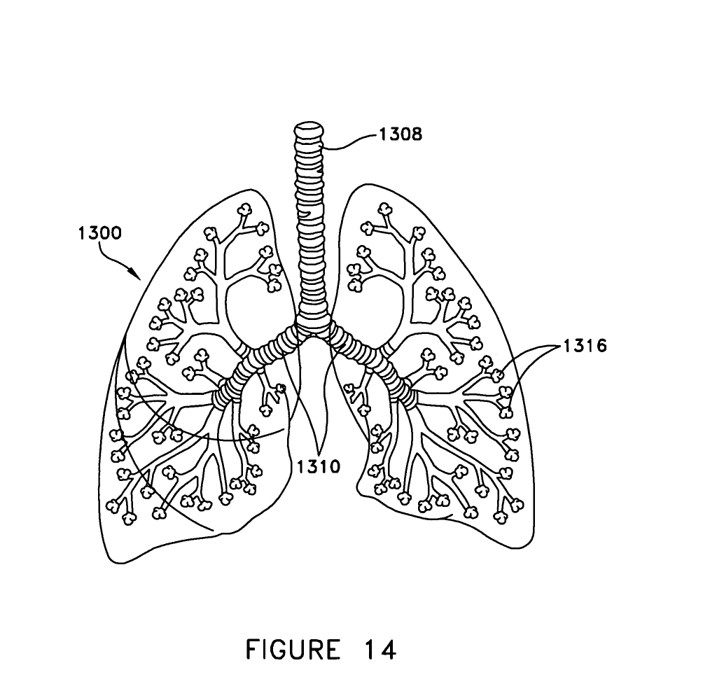

Looking at FIG. 12, and in an embodiment, there is shown another exemplary embodiment of a novel metered dose inhaler 100 having a canister holding portion 128 with a rounded contour 162. Extension portion 112 and canister holding portion 128 may be configured to articulate and telescope with respect to one another. Metered dose inhaler 100 is shown in the use configuration. FIG. 12A illustrates a cross-sectional view of metered dose inhaler 100 having canister holding portion 128 with a rounded contour 162. Additionally, portion 128 and portion 130 of telescoping articulation 126 may be configured to mate with one another to seal the back end of chamber 114.

As best shown in FIG. 7, there is illustrated a “V” shaped groove 164 on the sides of first portion 128, such that “V” shaped groove is adjacent medication canister 108. It should be appreciated that the present invention is not limited to a “V” shaped groove, but may include other configurations of openings in the sides of first portion 128, and may include only a single opening or more than two openings. In an embodiment, there are two “V” shaped grooves 164, one each side of first portion 128. “V” shaped grooves 164 give the user a place to grab canister 108 when removal is necessary, such as for replacement.

The side walls of existing inhalers generally extend nearly to the top of the canister. This supports the canister during use and storage. Very little of the canister protrudes from this portion of the sidewalls, and users often have a difficult time grabbing the canister and removing it. For example, users are known to use their teeth.

“V” shaped groove 164 also provides an increased tolerance between the size of first portion 128 and the size of canister 108. Existing canister holders have a large amount of clearance and may include some small ribs inside that bump into the sides of the canister. If the canister is larger the side walls of the holder tend to flex to accommodate this variation. In the present invention, first portion 128 may be configured as small a possible for storage inside chamber 114 of second portion 130. “V” shaped groove 164 accommodates variations in the diameter of canister 108. Generally, “V” shaped groove 164 allows first portion 128, the canister holder, to spread open to accommodate variations in size.

As best shown in FIG. 7, there is illustrated a lip 166 extending outwardly from at least a portion of the housing adjacent the first end thereof, and adjacent an outwardly extending portion of the canister when positioned in the first end of the housing. Lip 166 may provide people with small or weak hands something to hold onto when actuating canister 108. A common way to actuate a canister is to hold it between the thumb and middle finger and actuate by pinching. People with small or weak hands cannot either span this distance or do not have the strength to perform the pinch move. By placing lip 166 around at least a portion of the canister holder, a user may wrap his or her hand around first portion 128 and canister 108, with lip 166 resting above the top of a curled index finger. The user may then place his or her thumb over the top of canister 108 and press down with the thumb to actuate. This move may be practiced with existing canister holders. However, without lip 166, the tendency is for the holder to slip through the hand as it is actuated.

Looking now at FIG. 7B, there is a flat region 168 to accommodate a thumb and middle finger pinch move. Flat region 168, when canister holder 12 is placed into the closed position (see FIGS. 11 and 12) may form a snap fit with arcuate opening 139. This may help to keep first portion 128 from sliding out of second portion 130 while in the stored position.

What is claimed is:

1. A metered dose inhaler for administering medication, the metered dose inhaler comprising:
a housing having a first end and a second end, the first end being configured to hold a medication canister that is actuated to selectively administer medication through the housing and out of the second end;
a mouth-piece disposed adjacent the second end of the housing;
an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and
a one-way valve disposed adjacent the second end of the housing.

2. A metered dose inhaler in accordance with claim 1, wherein the housing is a single, integral body from the first end to the second end.

3. A metered dose inhaler in accordance with claim 1, wherein the housing includes a telescoping articulation between the first end and the second end.

4. A metered dose inhaler in accordance with claim 1, further comprising a flange disposed on the mouth-piece, between the chamber and the second end of the housing, and the flange configured a distance away from the second end of the housing to encourage the user to put teeth and lips around the mouth-piece, and to prevent occlusion of the mouth-piece with the teeth and lips.

5. A metered dose inhaler in accordance with claim 1, further comprising a peripheral edge extending inwardly from the housing adjacent to the medication canister, the peripheral edge configured to reduce a spray of the medication through the second end of the housing.

6. A metered dose inhaler in accordance with claim 1, further comprising a medication canister disposed within the first end of the housing.

7. A metered dose inhaler for administering medication, the metered dose inhaler comprising:
a housing having a first end and a second end, the first end being configured to hold a medication canister that is actuated to selectively administer medication through the housing and out of the second end;
a mouth-piece disposed adjacent the second end of the housing;
an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and
a flange disposed on the mouth-piece, between the chamber and the second end of the housing, and the flange configured a distance away from the second end of the housing to encourage the user to put teeth and lips around the mouth-piece, and to prevent occlusion of the mouth-piece with the teeth and lips.

8. A metered dose inhaler in accordance with claim 7, wherein the housing is a single, integral body from the first end to the second end.

9. A metered dose inhaler in accordance with claim 7, wherein the housing includes a telescoping articulation between the first end and the second end.

10. A metered dose inhaler in accordance with claim 7, wherein the flange includes a ridge of a raised, narrow strip of material disposed on the mouth-piece.

11. A metered dose inhaler in accordance with claim 10, wherein the ridge forms a continuous ring around a periphery of the mouth-piece.

12. A metered dose inhaler in accordance with claim 10, further comprising a peripheral edge extending inwardly from the housing adjacent to the medication canister, the peripheral edge configured to reduce a spray of the medication through the second end of the housing.

13. A metered dose inhaler in accordance with claim 7, further comprising a medication canister disposed within the first end of the housing.

14. A metered dose inhaler for administering medication, the metered dose inhaler comprising:
a housing having a first end and a second end, the first end being configured to hold a medication canister that is actuated to selectively administer medication through the housing and out of the second end;
a medication canister disposed at the first end of the housing, the medication selectively administered through the housing and out of the second end;
a mouth-piece disposed adjacent the second end of the housing;
an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and
a peripheral edge extending inwardly from the housing adjacent to the medication canister, the peripheral edge configured to reduce a spray of the medication through the second end of the housing.

15. A metered dose inhaler in accordance with claim 14, wherein the housing is a single, integral body from the first end to the second end.

16. A metered dose inhaler in accordance with claim 14, wherein the housing includes a telescoping articulation between the first end and the second end.

17. A metered dose inhaler in accordance with claim 14, further comprising a one-way valve disposed adjacent the second end of the housing, between the chamber and the mouth-piece.

18. A metered dose inhaler in accordance with claim 14, further comprising a flange disposed on the mouth-piece, between the chamber and the second end of the housing, and the flange configured a distance away from the second end of the housing to encourage the user to put teeth and lips around the mouth-piece, and to prevent occlusion of the mouth-piece with the teeth and lips.

19. A metered dose inhaler in accordance with claim 14, further comprising a medication canister disposed within the first end of the housing.

20. A metered dose inhaler for administering medication, the metered dose inhaler comprising:
a housing having a first end and a second end, the first end being configured to hold a medication canister that is actuated to selectively administer medication through the housing and out of the second end;
a mouth-piece disposed adjacent the second end of the housing;
an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user; and
a telescoping articulation between the first end and the second end of the housing, a first portion of the housing, between the articulation and the first end, having the medication canister therein, a second portion of the housing, between the articulation and the second end, having the mouth-piece and the extension, and the first portion and the second portion configured to articulate with respect to one another to slide together to position the second portion in a surrounding configuration with the first portion for storage, and to slide apart from one another and articulate with respect to one another, to position the mouth-piece of the second portion away from the medication canister within the first portion for use.

21. A metered dose inhaler in accordance with claim 20, wherein the first and second portions of the housings are configured to form a seal therebetween when the first portion and the second portion are slid apart from one another and articulated with respect to one another for use.

22. A metered dose inhaler in accordance with claim 20, further comprising a flange disposed on the mouth-piece, between the chamber and the second end of the housing, and the flange configured a distance away from the second end of the housing to encourage the user to put teeth and lips around the mouth-piece, and to prevent occlusion of the mouth-piece with the teeth and lips.

23. A metered dose inhaler in accordance with claim 20, further comprising a one-way valve disposed adjacent the second end of the housing, between the chamber and the mouth-piece.

24. A metered dose inhaler in accordance with claim 20, wherein the telescoping articulation includes at least one of the first portion and the second portion having a slot formed therein, and further comprises a pivot disposed within at least one of the slot of the at least one of the first portion and the second portion.

25. A metered dose inhaler in accordance with claim 20, wherein one of the first portion and the second portion includes a track formed therein, and the other one of the first portion and the second portion contains a pivot extending therefrom, corresponding to the track.

26. A metered dose inhaler in accordance with claim 20, wherein the pivot extends from the first portion, and the track is formed within the second portion.

27. A metered dose inhaler in accordance with claim 26, wherein the first portion defines openings therein adjacent to the pivot, and wherein the openings are configured to relieve pressure buildup in the chamber when medication is dispensed from the medication canister.

28. A metered dose inhaler in accordance with claim 20, further comprising a medication canister disposed within the first end of the housing.

29. A metered dose inhaler in accordance with claim 20, wherein the first portion and the second portion are configured to lock with one another to position the mouth-piece of the second portion away from one another for use.

30. A metered dose inhaler in accordance with claim 29, wherein the second portion includes an arcuate opening sized to mate with the housing of the first portion, and an attachment device including projections formed by the shape of the opening.

31. A metered dose inhaler in accordance with claim 20, wherein the first portion and the second portion are configured to lock with one another to position the second portion in a surrounding configuration with the first portion for storage.

32. A metered dose inhaler in accordance with claim 31, wherein the first portion and the second portion are sized for frictional engagement with one another to lock with one another.

33. A metered dose inhaler for administering medication, the metered dose inhaler comprising:
a housing having a first end and a second end, the first end being configured to hold a medication canister that is actuated to selectively administer medication through the housing and out of the second end;
a mouth-piece disposed adjacent the second end of the housing;
a telescoping extension forming a chamber within the housing, between i) where a nozzle of the medication canister is positioned when the medication canister is held by the first end of the housing, and ii) the mouth-piece, wherein the telescoping extension has a variable length from the medication canister to the mouth-piece, a maximum length of the telescoping extension configured to optimize delivery of the medication from the medication canister to lungs of a user, and a minimum length of the telescoping extension configured to optimize portability of the metered dose inhaler; and
a one-way valve disposed adjacent the second end of the housing, between the chamber and the mouth-piece.

34. A metered dose inhaler in accordance with claim 33, wherein the telescoping extension further comprises selectively engaging portions for locking the variable length of the telescoping extension at the maximum length.

35. A metered dose inhaler in accordance with claim 33, wherein the telescoping extension further comprises selectively engaging portions for locking the variable length of the telescoping extension at the minimum length.

36. A metered dose inhaler in accordance with claim 35, wherein one of the selectively engaging portions forms a recess therein, another one of the selectively engaging portions forms a protrusion therefrom, and the recess and the protrusion are configurable to selectively lock together with one another.

37. A metered dose inhaler in accordance with claim 33, further comprising a medication canister disposed within the first end of the housing.

38. A metered dose inhaler for administering medication, the metered dose inhaler comprising:
a housing having a first end and a second end, the first end being configured to hold a medication canister that is actuated to selectively administer medication through the housing and out of the second end;
a mouth-piece disposed adjacent the second end of the housing;
a telescoping extension forming a chamber within the housing, between i) where a nozzle of the medication canister is positioned when the medication canister is held by the first end of the housing, and ii) the mouth-piece, wherein the telescoping extension has a variable length from the medication canister to the mouth-piece, a maximum length of the telescoping extension configured to optimize delivery of the medication from the medication canister to lungs of a user, and a minimum length of the telescoping extension configured to optimize portability of the metered dose inhaler; and
a flange disposed on the mouth-piece, between the chamber and the second end of the housing, and the flange configured a distance away from the second end of the housing to encourage the user to put teeth and lips around the mouth-piece, and to prevent occlusion of the mouth-piece with the teeth and lips.

39. A metered dose inhaler in accordance with claim 38, wherein the telescoping extension further comprises selectively engaging portions for locking the variable length of the telescoping extension at the maximum length.

40. A metered dose inhaler in accordance with claim 38, wherein the telescoping extension further comprises selectively engaging portions for locking the variable length of the telescoping extension at the minimum length.

41. A metered dose inhaler in accordance with claim 38, wherein one of the selectively engaging portions forms a recess therein, another one of the selectively engaging portions forms a protrusion therefrom, and the recess and the protrusion are configurable to selectively lock together with one another.

42. A metered dose inhaler in accordance with claim 38, further comprising a medication canister disposed within the first end of the housing.

43. A metered dose inhaler for administering medication, the metered dose inhaler comprising:
a housing having a first end and a second end, the first end being configured to hold a medication canister that is actuated to selectively administer medication through the housing and out of the second end;
a mouth-piece disposed adjacent the second end of the housing;
a telescoping extension forming a chamber within the housing, between i) where a nozzle of the medication canister is positioned when the medication canister is held by the first end of the housing, and ii) the mouth-piece, wherein the telescoping extension has a variable length from the medication canister to the mouth-piece, a maximum length of the telescoping extension configured to optimize delivery of the medication from the medication canister to lungs of a user, and a minimum length of the telescoping extension configured to optimize portability of the metered dose inhaler; and
a peripheral edge extending inwardly from the housing adjacent to the medication canister, the peripheral edge configured to reduce a spray of the medication through the second end of the housing.

44. A metered dose inhaler in accordance with claim 43, wherein the telescoping extension further comprises selectively engaging portions for locking the variable length of the telescoping extension at the maximum length.

45. A metered dose inhaler in accordance with claim 43, wherein the telescoping extension further comprises selectively engaging portions for locking the variable length of the telescoping extension at the minimum length.

46. A metered dose inhaler in accordance with claim 43, wherein one of the selectively engaging portions forms a recess therein, another one of the selectively engaging portions forms a protrusion therefrom, and the recess and the protrusion are configurable to selectively lock together with one another.

47. A metered dose inhaler in accordance with claim 43, further comprising a medication canister disposed within the first end of the housing.

48. A metered dose inhaler for administering medication, the metered dose inhaler comprising:
a housing having a first end and a second end, the first end being configured to hold a medication canister that is actuated to selectively administer medication through the housing and out of the second end, and the first end having a v-shaped groove formed therein;
a mouth-piece disposed adjacent the second end of the housing; and
an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user.

49. A metered dose inhaler for administering medication, the metered dose inhaler comprising:
a housing having a first end and a second end, the first end being configured to hold a medication canister that is actuated to selectively administer medication through the housing and out of the second end;
a lip extending outwardly from at least a portion of the housing adjacent the first end thereof, and adjacent an outwardly extending portion of the canister when positioned in the first end of the housing;
a mouth-piece disposed adjacent the second end of the housing; and
an extension forming a chamber within the housing, between i) where a nozzle of the medication canister is positioned when the medication canister is held by the first end of the housing, and ii) the mouth-piece, wherein the extension has a length from the nozzle to the mouth-piece configured to optimize delivery of the medication from the medication canister to lungs of a user.
